SEQUENCE LISTING (5 sequences)

SEQ ID NO: 1 - DNA, 8 bp, Artificial Sequence
  8 Oligonucleotide combination for digit number 2
aaaacctg

SEQ ID NO: 2 - DNA, 4 bp, Artificial Sequence
  DNA segment for literal ibQ
gact

SEQ ID NO: 3 - DNA, 4 bp, Artificial Sequence
  DNA segment for literal ibP
tgca

SEQ ID NO: 4 - DNA, 4 bp, Artificial Sequence
  DNA segment for literal R
acgt

SEQ ID NO: 5 - DNA, 12 bp, Artificial Sequence
  DNA molecule concatenated from the DNA segment for literals ibQ, ibP and R
gacttgcaacgt

(19) United States
(12) Patent Application Publication
(10) Pub. No.: US 2005/0158717 A9
(43) Pub. Date: Jul. 21, 2005

(54) DNA computing method of providing solutions of theorem proving

(76) Inventors: Byoung Tak Zhang, Seoul (KR); Young Gyu Chai, Kyunggi-do (KR); Ji Yoon Park, Book-gu (KR); In Hee Lee, Seoul (KR)

Correspondence Address: HUNTON & WILLIAMS LLP, 1601 BRYAN STREET, ENERGY PLAZA - 30TH FLOOR, DALLAS, TX, 75201, US

(21) Appl. No.: 10/448,926

(22) Filed: May 30, 2003

(30) Foreign Application Priority Data
Jul. 8, 2002 (KR) 2002-0039320
Aug. 13, 2002 (KR) 2002-0047795

(51) Int. Cl. C12Q 1/68; G06N 3/12; G06F 19/00; G01N 33/48; G01N 33/50; G06F 15/18

(52) U.S. Cl. 435/6; 702/20; 706/13

(57) ABSTRACT

Disclosed is a DNA computing method of providing solutions of theorem proving with a resolution refutation. A positive literal of a clause is expressed as a base sequence while its negation is expressed as the complementary base sequence. The DNA molecules corresponding to clauses are hybridized with each other, followed by ligating the nicks of the hybrids. By use of PCR, a PCR product is obtained form with the ligated DNA molecules. The theorem proving is decided to be true if a perfect double strand of DNA is formed as measured by PAGE. The DNA molecules corresponding to the clause are of linear, branched or hairpin structures.

### TECHNICAL FIELD

The present invention relates, in general, to DNA computing and, more particularly, to a DNA computing method of providing solutions of theorem proving with a resolution refutation.

### BACKGROUND ART

Beyond conventional, sequential, irreversible silicon computing. LM computing, also known as molecular computing, is a new computational paradigm that harnesses biological molecules to solve computational problems, with the promise of massive parallelism to find solutions to huge problems by invoking a parallel search when enough DNA information is given. Research in this area began with an experiment by Leonard Adleman, in 1994 by using the tools of molecular biology to solve a hard computational problem.

Current silicon computing uses binary bits to write information in code where binary numbers 1 and 0 correspond to electric states “ON” and “OFF”. The basic calculation of information processing in silicon computing consists of logical product (AND calculation: e.g., 1 and 1=1; 1 and 0=0; 0 and 0=0) and logical sum (OR calculation: e.g., 1 or 1=1; 1 or 0=1; 0 or 0=0).

By contrast, DNA computing represents any information with synthetic DNA molecules consisting of four basic bases adenine (A), guanine (G), thymine (T) and cytosine (C). Instead of using electrical impulses to represent bits of information, DNA computers use the chemical properties of these molecules by examining the patterns of combination or growth of the molecules or strings. For instance, when the digit number “2” is represented, silicon-based computers use electric states “ON” and “OFF” corresponding to the 8-bit combination 00000010. In contrast, DNA-based computers employ the bases in the 8-oligonucleotide combination AAAACCTG.

According to the father of DNA computing, Adleman, computing by representing any information in DNA base sequences enjoys thee following advantages thanks to the massive parallelism of the biochemical reactions on DNA molecules:
(1) DNA models, based on molecular biology, can process 10¹⁴ bits per second, which is 100 times faster than the fastest silicon-based supercomputer ever developed thus far.
(2) DNA models can perform 2×10¹⁹ operations per joule, which is one-billionth the energy an ordinary desktop computer consumes.
(3) The information density of DNA is much greater than that of silicon: 1 bit can be stored in approximately one cubic nanometer. Other storage media, such as videotapes, can store 1 bit in 10,000,000,000,000 cubic nanometers.

Huge information can be represented in logic which can be processed. As well known in the computation and artificial intelligence fields, logic is used as a method for expressing the most fundamental knowledge and inferring new knowledge from the preexisting knowledge known to be true.

In order to enable DNA-based computers to process a huge amount of information of real life and infer new knowledge with logic, it is important to develop logic expressions and processing methods. Logic expressions consist of true and false symbols for expressing truth or falsehood, Boolean variables which can take only T (true) or F (false) as their values, and connectives such as logical sum ( [symbol] ), logical product ( [symbol] ), negation ( [symbol] ), implication ( [symbol] ) and equality ( [symbol] ). For the processing of logic, such concepts or methods as propositional logic, propositional calculus, predicate logic, predicate calculus, inference, resolution and refutation are mainly used.

In the automated reasoning field which is one of the artificial intelligence fields, research has been made on silicon computer-based logic processing methods for representing knowledge of real life and eliciting new 2 knowledge from the known knowledge through inference. Theorem proving, one of the problems which has been under study in the field, is a method for logical reasoning which determines whether or not knowledge newly obtained based on already known knowledge coincides with preexisting knowledge or whether or not new knowledge can be obtained from preexisting knowledge through a logical procedure. The automated reasoning field sets the goal of letting computers automatically perform such processes so as to endow the computers with automated information processing capability. An expert system is an example in which such technology is applied to real life.

There are two methods of eliciting a solution to theorem proving: a deductive method in which, starting from a preexisting knowledge (hereinafter referred to as “theory”), a new knowledge (hereinafter referred to as “theorem”) is obtained through rounds of deduction; and a refutation method in which the negation of a theorem is added to a theory set and research is made on whether inconsistency happens in the theory set. The deduction method is known to have no assured measures for resolving a theorem from a theory. In contrast, the refutation method makes it possible to give a solution to theorem proving only by repetitively applying reasoning rules to theory and theorem sets. That is, computers into which available reasoning rules are inputted can automatically solve theorem proving problems. Therefore, extensive research has been made on the refutation method in the automated reasoning field.

Because resolution is not only a reasoning method of always eliciting logically true results, but gives simple and mechanical rules suitable for computers, it has been used to find solutions to theorem proving in computers. However, it is necessary to express the theory and theorem in a certain form, that is, a conjunctive normal form.

Prior to defining the conjunctive normal form, explanations are needed for some terms. The term “literal” as used herein means a most fundamental unit of a logical formula, and may be subject to a positive or a negative literal. Positive literals consist of Boolean variables themselves while negative literals are expressed as negations of Boolean variables. The term “clause” is defined as a formula in which positive or negative literals are connected to one another only through a logical sum. The conjunctive normal form means a formula in which clauses are connected to one another only through a logical product, and is expressed as a set of clauses on a computer. According to research results, it is known that all logical formulas can be expressed in a conjunctive normal form by making use of the distribution law [P [symbol] (Q [symbol] R) [symbol] (P [symbol] Q) [symbol] (P [symbol] R)] and the De Morgan law [ [symbol] (P [symbol] Q)] [symbol] [ [symbol] P [symbol] [symbol] Q].

Following is how to calculate logical formulas which are expressed in a conjunctive normal form and to which resolution is applied assuming that a positive literal is subject to one of two clauses and its negation to the other clause. After removals of the two contradict literals from the two clauses, the remains can be connected via a logical sum to draw a clause. It is demonstrated that the obtained clause is the result of the logically correct derivation from, the two clauses. For instance, two clauses P [symbol] Q and [symbol] P [symbol] R are derived into Q [symbol] R through resolution. The newly drawn clause Q [symbol] R is called resolvent.

Where a theorem proving problem is solved by a resolution refutation, the presence of inconsistency produces a conjunctive normal form of P [symbol] [symbol] P which is expressed as two clauses, P and [symbol] P, on computers. If the resolution continues to be applied to the two clauses, the resulting resolvent is derived into a clause to which no literal is subject, called an “empty clause”. In the resolution refutation, the empty clause is construed as a signal of inconsistency. Proof is an orderly arrangement of the clauses and resolvents which are used until an empty clause is produced.

A simple example of the resolution refutation is seen below. Given is a theory set of “if it rains, then the ground is wet.”, “it rained.” Let us prove that a proposition of “the ground was wet” is the conclusion which can be drawn from this theory set. If the proposition of “it rains” is a expressed as a Boolean variable R and the proposition of “the ground is wet” as a Boolean variable E, the proposition of “if it rains, then the ground is wet” is expressed as R [symbol] E. This is known to be equivalent to [symbol] R [symbol] E. Thus, the theory set is { [symbol] R [symbol] E, R}. The negation of the logical formula to be proved, expressed as [symbol] E, is added to the clauses [symbol] R [symbol] E and R to which resolution is then applied to draw E. Resolving an clauses E and [symbol] E results in an empty clause. Therefore, it is proven that the original logical formula is the conclusion which can be logically drawn from the theory set.

As seen above, the resolution refutation is a sequential repetition of a procedure in which a resolution is applied to two clauses to draw a new resolvent. In a theorem proving process, the complexity of proof grows exponentially with given clauses. Because of their limitations to a sequential calculation, silicon-based computers can test logically correct proofs one by one. Hence, there is needed a strategy for such discreet selection of clauses to which resolution is applied as to test as few numbers of proofs as possible. To this end, a breadth-first strategy and a depth-first strategy were suggested. In addition, as an approach to reduce the selection range of the clauses to which resolution is applied, linear resolution, semantic resolution or etc. was developed. Despite all these strategies, however, no exceptional advance in performance has been achieved on silicon-based computers.

A solution to this problem is to parallelize the theorem proving process of drawing a resolvent from two clauses. The massive parallelism of DNA molecular reactions for implementing parallel theorem provers makes it possible to produce resolvents from many clauses simultaneously. Accordingly, the massive parallelism of DNA molecular reactions not only exceptionally reduces the time period which it takes to perform a theorem proving process, but enables various theorem proving processes to be implemented at the same time. In contrast to silicon-based computers where the computing time period increases exponentially with the number of clauses, DNA-based computers can give solutions to theorem proving in an exceptionally short period of time. Moreover, if theorem proving problems are solved by experiments, DNA molecules can be used without digitalizing the biological data which are enriched due to great advances in the human genome project.

Among the problems which are difficult to effectively solve with conventional computers, the satisfiability problem exists. This problem requires calculations for the conjunctive normal form. It is known that all of the solve-hard problems can be converted into satisfiability problems. That is, finding solutions to satisfiability problems is finding solutions to the problems which are reversibly converted into the satisfiability problems. Intensive research has been done on these problems by use of the above-mentioned DNA computing, leading to the following results in which clauses in conjunctive normal form are represented with DNA and processed.

In the case that only one of the two literals constituting a clause is a negative literal, the clause Corresponds to a simple “IF-THEN” rule and can be represented by a double strand of DNA (P. Wasiewicz, T. janczak, j. J. Mulawka, and A. Rlucienniczak, The inference based on molecular computing, Cybernetics and Systems, 31:283-315, 2000). However, this method has a disadvantage in that expression is impossible when the nun er of literals in a clause exceeds two.

Likewise, after the condition of “IF” and the conclusion of “THEN” in each “IF-THEN” rule are given different sequences, single strands of DNA in which complementary sequences are connected to the sequences given to the condition and the conclusions are used to express a set of “IF-THEN” rules (K. Sakamoto, D. Kiga, K. Komiya, H. Gouzu, S. Yokoyama, S. Ikeda, H. Sugiyama, M. Hagiya, State transition by molecules, In Proceedings of 4^{th} DIMACS Workshop on DNA Based Computers, 1998). In this expression method, a set of “IF-THEN” rules is expressed by a sequence in which the sequences given to the rules are connected in a line. However, where the “IF” condition of one rule corresponds to the “THEN” conclusion of another rule, many complementary sequences exist in a single strand of DNA so that they are highly likely to undesirably bind to each other. Additionally, this method cannot express a clause containing three or more literals. Between literals contained in a clause, sequences that different restriction enzymes recognize are inserted to express the clause (S. Kobayashi, T. Yokomori, G Sampei, and K. Mizobuchi, DNA implementation of simple horn clause computation. In Proceedings of the IEEE International Conference on Evolutionary Computation, 1997). However, there are a limited number of restriction enzymes in the natural world so that the method cannot express the clause which contains literals more than restriction enzymes. Furthermore, reaction conditions for restriction enzymes are so different that high complexity is loaded on experiment.

Methods for expressing highly complex logical formulas with DNA and processing them are under study. In particular, many attempts have been made to use DNA to express literals and clauses of logical formulas, but is limited to the expression of logical formulas or “IF-THEN” rules, research on chain reactions, or solutions to the Hamiltonial path problem or satisfiability problems.

### DISCLOSURE OF THE INVENTION

With the prior problems in mind, the present invention has an object of providing a DNA computing method using a resolution refutation, in which theorem proving can be solved in a parallel manner and experimentally implemented in order to accomplish the above object, the present invention provides a DNA computing method of providing solutions of theorem proving, comprising the steps of: representing logical clauses for theorem proving with DNA molecules; synthesizing the DMA molecules; chemically reacting the synthetic DNA molecules; and deciding solutions to the theorem proving based on the result of the chemical reaction.

In accordance with an, embodiment, the representing step comprises encoding a positive literal of the clauses with a base sequence, the negation of the positive literal with the complementary base sequence, and a clause with a single strand of DNA or a branched form of DNA.

### BRIEF DESCRIPTION OF THE DRAWINGS

The above and other objects, features and other advantages of the present invention will be more clearly understood from the following detailed description taken in conjunction with the accompanying drawings, in which:

FIGS. 1 and 2 are schematic views illustrating theorem proving problems;

FIG. 3 is a flowchart illustrating a solution to theorem proving problems by use of a resolution refutation;

FIGS. 4A and 4B are schematic views illustrating two ways of representing conjunctive normal forms with DNA;

FIG. 5 is a view illustrating the representation of Boolean variables commonly used in the two representing ways of FIGS. 4A and 4B;

FIG. 6 is an electrophoretogram showing PCR products obtained when the resolution refutation DNA computing of the present invention is applied to the theorem proving problem of FIG. 1 by use of the conjunctive normal form of FIG. 4A;

FIG. 7 is an electrophoretogram showing PCR products obtained when the resolution refutation DNA computing of the present invention is applied to the theorem proving problem of FIG. 2 by use of the conjunctive normal form of FIG. 4B; and

FIGS. 8 and 9 show examples of base sequences designed to represent logical formulas according to the present invention.

### BEST MODE FOR CARRYING OUT THE INVENTION

The application of the preferred embodiments of a DNA computing method according to the present invention is best understood with reference to the accompanying drawings, wherein like reference numerals are used for like and corresponding parts, respectively.

Solutions to theorem proving are drawn by connecting a literal of one clause to the negation of the literal of the other clause in a conjunctive normal form to produce a resolvent in accordance with conventional computation whereas by hybridizing a certain base sequence with its complementary sequence in the present invention. Additionally, a final result through the resolution refutation is determined by the production of an empty clause in conventional methods while by the production of a perfect double strand of DNA which is verified by PCR in the present invention. In giving solutions to theorem proving according to the DNA computing of the present invention, each DNA molecule is used to express a clause and resolution results can be obtained through the parallel and spontaneous reactions of the molecules used.

The expression of conjunctive normal forms according to the present invention is based on the finding that, when complementary sequences are given to positive/negative literals in the conjunctive normal forms, the procedure of removing the positive/negative literals from the two clauses to draw a resolvent can be represented with the annealing reaction between DNA molecules corresponding to clauses, characterized by (1) giving a certain DNA sequence to a positive literals, (2) expressing the negative literal with the DNA sequence complimentary to the DNA sequence corresponding to the positive literal, and (3) representing one clause with at least one strand of DNA molecules. DNA strands corresponding to clauses may take linear forms, hairpin structures or branched forms.

In conventional computing methods of providing solutions of theorem proving with the resolution refutation, all clauses are stored in memory devices and combined in every possible case to produce resolvents which are then reduced in number while researching on the drawing of empty clauses. In contrast to conventional methods, the DMA computing method of the present invention provides very efficient solutions to theorem proving.

In more detail, solutions to theorem proving according to the present invention are provided by synthesizing DNA molecules corresponding to clauses, mixing the synthetic DNA molecules in a test tube and performing biochemical experiments for confirming the production of empty clauses, that is, hybridization, ligation, PCR and electrophoresis. Furthermore, the DNA computing method according to the present invention is characterized by the ability to process in a parallel manner the clauses of a logical formula represented in conjunctive normal form.

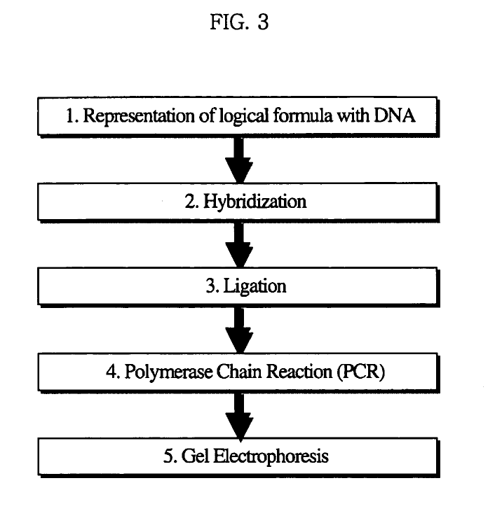

With reference to FIG. 3, a flow chart shows a procedure for providing a solution to theorem proving by use of the resolution refutation in accordance with the DNA computing method of the present invention. As seen in FIG. 3, a solution to theorem proving is drawn according to the present invention as follows:
(1) Encode each positive literal with a predetermined DNA sequence while representing its negative literal as the complementary DNA sequence. Represent each clause with a combination of DNA sequences and mix DNA molecules corresponding to clauses.
(2) Hybridize the molecules, each representing a clause, to perform a resolution.
(3) Ligate nicks of the DNA hybrids with the aid of ligase.
(4) Amplify the ligation products by PCR.
(5) Electrophorese the PCR products on gel to determine their sizes.

In step (1), the representation of clauses with DNA can be largely divided into two: how to represent Boolean variables or literals of clauses and then clauses by use of them.

According to the present invention, a positive literal is represented with a predetermined base sequence of a suitable length while its negation corresponds to the complimentary sequence. As for the DNA sequences for representing literals, their lengths and base arrangements, though not specifically limited, are preferably selected so as to hybridize with their complementary sequences at suitable temperatures. Usually, the DNA sequences are approximately 10-30 mer in length. In consideration of hybridization conditions, the sequence is determined as to GC content, base arrangement, and length. For convenience of explanation, a base sequence which is suitable for hybridization is called a predetermined base sequence in this specification. Those skilled in the art can determine whether a base sequence is proper for hybridization under specific conditions or not.

Following are four conditions which are considered when determining base sequences. That is, (1) Base sequences corresponding to different positive/negative literals must be as inconsistent as possible. (2) Base sequences corresponding to different positive/negative literals hybridize with each other at as low possibility as possible. (3) Base sequences corresponding to different positive/negative literals have as uniform melting points and GC contents within the range of hybridization conditions required as possible. (4) In each of the base sequences corresponding to different positive/negative literals, as few the same bases as possible are arranged in a tandem manner. It is very important to solutions to theorem proving problems to design sequences satisfying the above conditions. DNA molecules may form hybrids in non-Watson-Crick complementary manners or may be not amplified in desired intramolecular regions for various reasons such as base sequence homology, intermolecular bonding, and secondary structure. Algorithms or programs for designing optimal DNA sequences can be applied for securing industrially useful genes in addition to solutions to theorem proving.

Base sequences used in the present invention can be designed by a base sequence generator such as multiobjective genetic algorithm-based base sequence generator NACST/Seq (Shin, S.-Y. Kim, D.-M, Lee, I.-H., and Zhang, B.-T., Evolutionary sequence generation for reliable DNA computing. Proceedings of 2002 Congress on Evolutionary Computation, IEEE press, 2002).

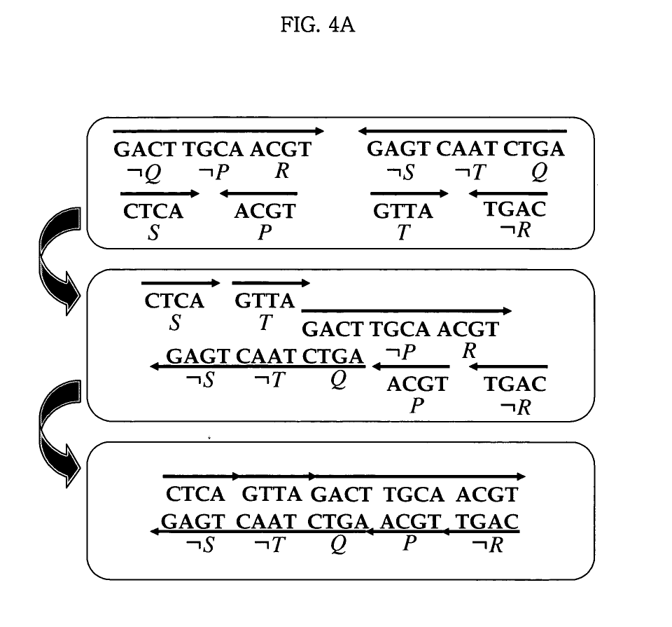

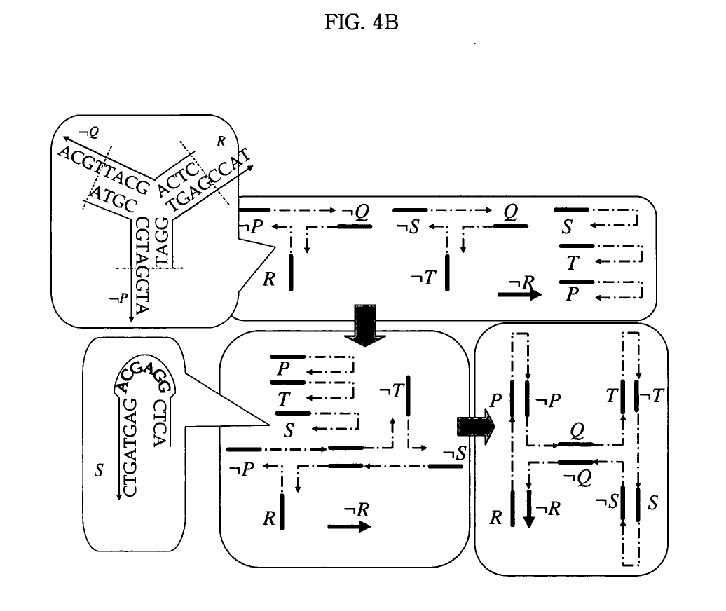

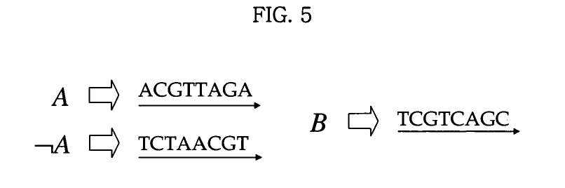

In the present invention, a clause is represented by a single DNA molecules formed from DNA segments corresponding to literals. The representation of clauses nay be done largely in two manners one is to concatenate DNA segments corresponding to literals into a single strand of DNA corresponding to the clause. For example, as shown in FIG. 4A, when a clause with literals [symbol] Q, [symbol] P and R are given DNA segments GACT, TGCA and ACGT for the liberals, respectively, the clause is represented as GACTTGCAACGT concatenated from the DNA segments.

The other representation method is to use hairpin structures of DNA or branched DNA. In the case of branched DNA, a stretch of base sequences representing a literal is positioned at the end of each branch. The branched DNA molecule is formed by combining three or more strands of DNA in such a way that one strand partially binds to other two strands, which is illustrated by the upper-left box in FIG. 413. As for the hairpin structure, base paring occurs beg e distant complementary segments in one strand. In accordance with the present invention, a branched DNA corresponding to a clause has as many arms as literals of the clause. Each arm has a sticky end corresponding to each literal. Sticky ends for the positive and negative literals of one variable are complementary. Every clause is not represented in the same DNA structure. According to the number of the literals of clauses and whether each clause is the goal clause to be proved, representation for clauses takes different forms. For instance, where a clause with one literal is the goal clause to be proved in a theorem proving problem, it is encoded with a linear single strand. On the other hand, for a clause with one literal except the goal clause, it is represented with a hairpin molecule having a sticky end in which a stretch of bases corresponding to the clause are arranged. Each clause with two or more literals is denoted by a two- or more armed branched molecule. Such representation manners are illustrated in FIG. 4B.

The representation of clauses with branched EMA makes the result of the hybridization step free from the arrangement of literals in the clauses. In addition, the use of branched molecules in the representation of clauses gives feasibility to the selection of PCR primers. That is, when an empty clause is drawn, it will start with a goal sequence and end with its negation since clauses are either branched molecules or hairpin molecules except for the goal. Therefore, at the PCR step, the sequence for the literal of the goal and its complementary sequence are used as printers. On the other hand when representing clauses with linear single strands of DNA, the design of PCR primers must depend only on predicting which DNA hybrid is formed

In a conjunctive normal form, the procedure of deriving a resolvent from a clause with a positive literal and a clause with the negation of the positive literal, that is, resolution between the two clauses is denoted as the hybridization of two DNA sequences corresponding respectively to the two clauses. When an empty clause is drawn, a perfect double strand of DNA is obtained. In this case, the PCR product corresponds to the sum of the lengths of the DNA sequences for the total literals. The term “a perfect double strand of DNA” as used herein means a DNA molecule in which a base sequence in one strand binds to its complementary sequence in the other strand after hybridization and ligation, since a base sequence for a positive literal is contained together with a base sequence for a negative literal in a reaction container.

In accordance with the present invention, the production of an empty clause results in a double strand of DNA consisting of two DNA strand upon linear implementation and in a double strand of DNA consisting of one DNA strand upon hairpin or branched implementation (see FIGS. 4A and 4B).

A better understanding of the method of encoding clauses and the DNA computing method of providing solutions of theorem proving with the resolution refutation may be obtained in light of the following examples which are set forth to illustrate, but are not to be construed to limit the present invention.

### EXAMPLE

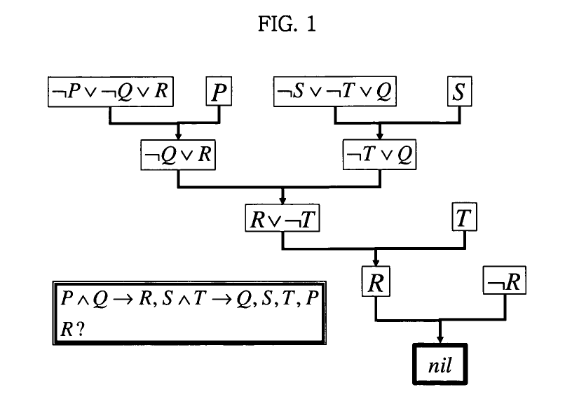

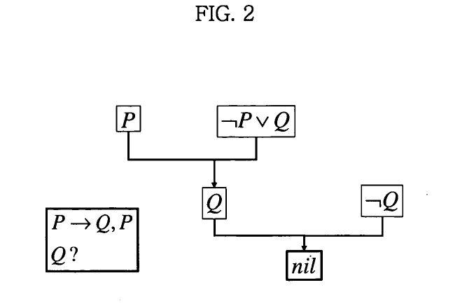

An example of the theorem proving problem to be solved is given as shown in FIGS. 1 and 2. In the following examples, the DNA computing of the present invention is used to prove that when the set of formulas {P [symbol] Q [symbol] R, S [symbol] T [symbol] Q, S, T, P} is given, R is true and that when the set of formulas {P [symbol] Q, P}, Q is true. According to logic knowledge, the formula {P [symbol] Q [symbol] R}can be converted into the clause { [symbol] P [symbol] [symbol] Q [symbol] R}. Likewise, {S [symbol] T [symbol] R} can be expressed as { [symbol] S [symbol] [symbol] R}. Thus, the theorem proving problem shown in FIG. 1 corresponds to deciding whether an empty clause can be drawn by applying resolution to the conjunctive normal form [( [symbol] P [symbol] [symbol] Q [symbol] R) [symbol] ( [symbol] S [symbol] [symbol] T [symbol] R) [symbol] S [symbol] T [symbol] P [symbol] [symbol] R ]. The same is true of the theorem proving problem shown in FIG. 2. That is, because {P [symbol] Q} can be expressed as { [symbol] P [symbol] Q}, resolution is applied to the conjunctive normal form [( [symbol] P [symbol] Q)) [symbol] P [symbol] [symbol] Q] to produce an empty clause.

1. Representation of Clauses on Linear DNA

In this example, each literal was represented with 15 mer ssDNA which was designed by NACST/Seq in consideration of the aforementioned four conditions. Concrete procedures of designing base sequences by use of NACST/Seq are as follows.

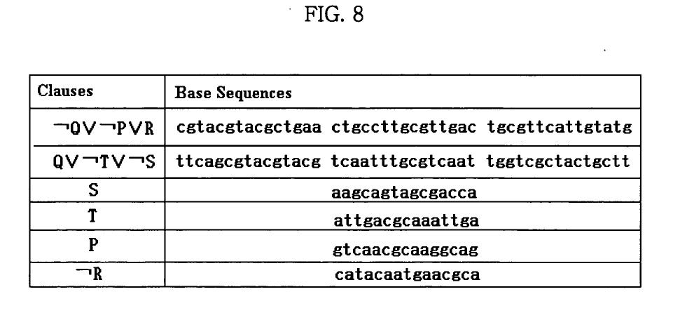

After determining the length and number of base sequences to be produced, a predetermined number of individuals are produced arbitrarily. Each individual denotes a set of a predetermined number of base sequences. Next, suitability of the individuals to each of the four conditions is calculated. This calculated suitability has great influence on the selection of the individuals which will be used in the next generation. In this regard, individuals with higher suitability are designed to be selected at a higher possibility. Two individuals taken from the selected individuals are subjected to crossover operations to produce two new individuals with predetermined probability and copied to the next generation. The other individuals are transferred, as they are, into the next generation. Then, a mutation operation is performed on randomly selected individuals at arbitrary positions with a predetermined possibility. Individuals produced through this procedure form individual sets in the next generation. The individual sets are repetitively subjected to selection an crossover, mutation calculation at a predetermined number of rounds. In the course of such processes, sets of base sequences corresponding to individuals with low suitability are excluded during the selection process and finally disappear, and individuals with high suitability survive users can select base sequence sets from the survivals. Sequences for clauses are given in FIG. 8. The produced DNA takes linear forms instead of branched forms.

2. Representation of Clauses on Hairpin DNA

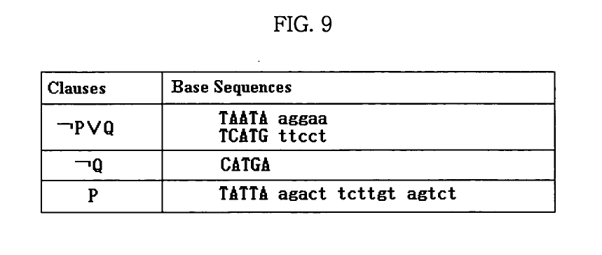

In this example, each literal was encoded with a characteristic DNA sequence of 5 mer. In case of branches, each branch has a double strand which is 5 bp long with the sticky end of a single strand. A hairpin DNA is structured to have a double strand of 5 bp and a loop head of 6-mer. Base sequences were designed to satisfy the problem of FIG. 2 by use of NACST/Seq. The designed sequences are given in FIG. 9. In the sequence of FIG. 9, parts corresponding to literals are represented by capitals, Turning to the problem of FIG. 2, the negation of the theorem to be proved is denoted as [symbol] Q. This was represented without use of branches or hairpin structures.

3. DNA Computing of Providing Solutions of Theorem Proving

FIGS. 1 and 2 show procedures of solving theorem proving problems with the resolution refutation when logical formulas and theorems are given. In FIG. 3, an experimental procedure of obtaining solutions to theorem proving by use of the DNA computing through the resolution refutation are illustrated. The experimental results for the solutions to theorem proving problems were found to be the same even though either of the two representation ways was taken. Experimental procedures for the two representation ways are schematically illustrated in FIGS. 4A and 4 dB.

In the present invention, in order to identify the final solutions of linear DNA and hairpin DNA, the procedure from hybrid formation to gel electrophoresis is performed under the same conditions.

1) Hybridization

100 pmol of each of the oligomers of FIG. 8 or 9 encoding clauses were mixed in a final volume of 20 μl in one vial. Using a PCR machine, initial denaturation was achieved at 95° C. Temperature was gradually lowered from 95° C. to 16° C. at a decreasing rate of 1° C./min to bind complementary sequences to each other. This hybridization can be applied to a medically useful DNA microarray chip which can probe gene expression and diagnose diseases. In more detail, the DNA microarray chip has a glass substrate on which tens of thousands of cDNA are immobilized. RNA from patients of interest and normal persons are amplified by RT-PCR into cDNA. Upon amplification, cDNAs from patients and normal persons are dyed with different fluorescent agents and combined with the cDNAs immobilized on the chip to detect the disease.

2) Verification of Formation of Empty Clause

When repetitive application of resolution to a conjunctive normal formula results in the formation of an empty clause, the DNA molecule obtained by the hybridization step has a double strand structure amplified.

Whether the DNA molecule is of a perfect double strand structure or not can be confirmed through the following processes.

2-1 Ligation

In order to ligate nicks within single strands of the hybrid obtained, it was mixed with a T4 DNA ligase in a reaction buffer (50 mM Tris-HCl pH 7.8, 10 mM MgCl₂, 5 mM DTT, 1 mM ATP, and 2.5 μg/ml BSA) for a total volume of 10 μl. This was incubated at 16° C. for at least sixteen hours to give a stable ligation product.

2-2 Polymerase Chain Reaction

In the case of connecting literals in a line, the amplification of the final solution was perform in a PCR machine to produce a perfect double strand of DNA corresponding to an empty clause as depicted in the scheme of FIG. 4A. Together with the 100 pmol of S and R primers, the ligate and 1 U of Tag polymerase was added to a PCR reaction buffer to give a total volume of 50 μl. After being initiated by denaturing the DNA molecules at 94° C. for 4 min, amplification was performed with 25 cycles of PCR, each consisting of denaturation at 94° C. for 30 sec, annealing at 58° C. for 30 sac and extension at 72° C. for 30 sec, followed by an additional extension at 72 *C for 10 min.

2-3 Polyacrylamide Gel Electrophoresis (PAGE)

The PCR products thus obtained were run on 15% polyacrylamide gel across which an electric field of 60 V was applied. After being dyed with EtBr, the PCR product of interest was found to be 75 bp in size on a UV illuminator as measured with reference to a DNA marker run together.

When branched or hairpin structure DNA is used to represent clauses, as seen in FIG. 4B, a perfect double strand of DNA corresponding to an empty clause starts with a goal sequence and ends with its negation. Therefore, at the PCR step, sequences corresponding to Q and Q are used as primers.

3) DNA Computing Results

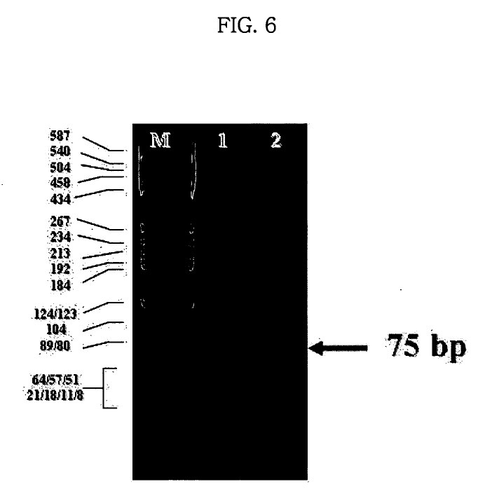

With reference to FIG. 6, there is an electrophoretogram obtained after linear ileaentation. In this example, a conjunctive normal form was expressed with five Boolean variables, each being set to have a size of 15 mer. Thus, the perfect double strand of DNA, which corresponds to the empty clause in this example, is 75 bp in total length. This was identified as a band as seen on PAGE of FIG. 6. Therefore, it comes to a conclusion that the theorem R can be resolved from given theories by use of the DNA computing of the present invention. This result is consistent with that obtained through the sequential, repetitive generation of resolvents (FIG. 1).

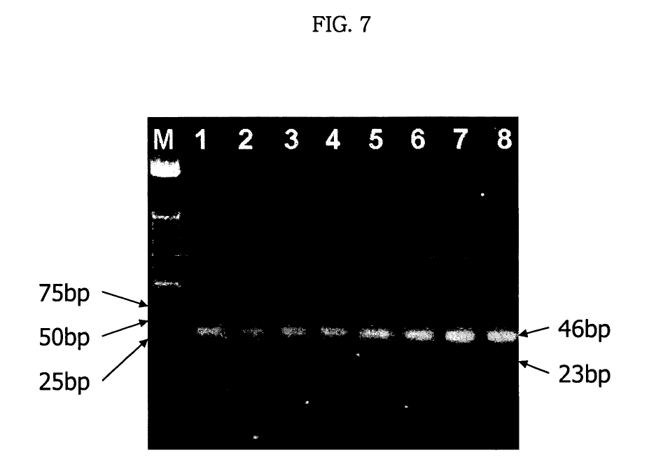

FIG. 7 shows electrophoresis results of liberals represented on branched or hairpin structure DNA. In this example, a conjunctive normal form is expressed with two Boolean variables, each being 5 mer in size. The clause { [symbol] P [symbol] Q} is expressed as a two-armed branch structure, whose double strand has a length of 5 hp. Thus, the DNA molecule corresponding to the empty clause will be a single strand of 46 mer or a double strand of 23 bp after ligation. In the case of the double strand, PCR will result in a double strand of 46 bp. These 46 mer (23 bp) or 46 bp DNA bands can be seen in the electrophotogram of FIG. 7. That is, it combs to a conclusion that Q can be logically resolved from given theories, which is consistent with the result obtained through the sequential, repetitive generation of resolvents (FIG. 2).

In another embodiment of the present invention, solutions of theorem proving can be automatically implemented on one chip. All of the processing steps including mixing, encoding, hybridizing, ligating, PCR, and electrophoresis steps can be feasibly performed on one chip, thereby finding solutions to theorem proving problems with higher rapidity, convenience and ease.

In a further embodiment, the DNA computing of the present invention can be applied to DNA microarray technology for disease diagnosis in which the expression extent of a gene relating to a disease of interest is probed. In this regard, the relationship between the expression of the disease-related gene and the attack of the disease can be described in a logical formula such as an “IF-THEN” rule. For example, supposing that if genes A and B are expressed at higher levels and a gene D expressed in a lower level in a person than in normal persons then he is diagnosed to suffer from a disease D. This can be expressed as the logical formula {(A [symbol] B [symbol] [symbol] C) [symbol] D} which can be then converted into the conjunctive normal form { [symbol] A [symbol] B [symbol] C [symbol] D}. The literals A, B, C are Boolean variables representing the proposition that. “genes A, B, C are expressed in higher levels in a patient than in normal person” while the literal D is a Boolean variable representing the proposition that “the patient suffers from a disease D”. Deciding whether the patient suffers from the disease D when his gene expression level is measured to be higher for genes A and B and lower for gene C than normal persons is equivalent to logically resolving D from the theory set { [symbol] A [symbol] [symbol] B [symbol] C [symbol] D, A, B, [symbol] C}. When this theorem proving problem is solved with the resolution refutation, an empty clause is drawn. Therefore, if a chip where all of the processing steps including mixing, encoding, hybridizing, ligating, PCR and electrophoresis can be implemented is inputted with rules of diagnosing a disease of interest and added with proper theories from the gene expression information of the patient, the resolution refutation leads to seeing whether the patient suffers from the disease. In addition, if the hybridizing step is performed in a separate compartment for each disease on the chip, different diseases or various patients can be diagnosed on only one chip. This is the difference from conventional diagnosis chips which can diagnose only one disease for only one patient.

### INDUSTRIAL APPLICABILITY

As described above, all logical formulas can be represented in conjunctive normal forms which can be very effectively processed in a parallel manner by the DNA computing of the present invention. Therefore, based on massive parallelism, the DNA computing of the present invention can be applied to the finding of solutions to complex theorem proving problems by use of a resolution refutation. For example, the procedure of solving theorem proving problems using the resolution refutation according to the DNA computing of the present invention can be applied to disease diagnosis and decision support systems. Thanks to massive parallelism, the DNA computing of the present invention allows theorem proving problems to be solved with a simple experimental procedure, and thus is easy to implement on chips. Further, the DNA computing of the present invention is useful for the construction of point-of-care diagnosis systems which are advanced versions of intelligence chips. Diagnosis results vary depending on DNA information, health state and life style. Information of each individual is stored in intelligent chips for diagnosis and investigated through hybridization with that of patients to develop diagnosing reagents which are the most suitable for each individual.

Generally well-known problems such as Horn clause have been solved by computer algorithms. These are proved in computer calculation methods, but not in experiments. Realization of computer simulation into experimental results of biochemical reactions and its proving procedure in vitro have been not seen ever. Such research enables theorem proving problems to be resolved in parallel manners to bring about effective calculation. When considering the basic concept that DNA computing is accomplished through molecular biological experiments, the present invention is expected to provide a great contribution to the industry.

1. A DNA computing method of providing solutions of theorem proving, comprising the steps of:
representing logical clauses for theorem proving with DNA molecules;
synthesizing the DNA molecules;
chemically reacting the synthetic DNA molecules; and
deciding solutions to the theorem proving based on the result of the chemical reaction.

2. The DNA computing method as defined in claim 1, wherein the representing step comprises encoding a positive literal of the clauses with a base sequence, encoding the negation of the positive literal with the complementary base sequence, and encoding a clause with a single strand of DNA.

3. The DNA computing method as defined in claim 2, wherein the base sequences are designed by use of a multiobjective functional genetic algorithm.

4. The DNA computing method as defined in claim 1, wherein the chemical reacting step comprises:
hybridizing the DNA molecules corresponding to clauses with each other;
ligating the nicks of the hybrids; and
performing polymerase chain reactions to give a PCR product with the ligated DNA molecules serving as templates.

5. The DNA computing method as defined in claim 1, wherein the deciding step comprises determining whether a perfect double strand of DNA is formed, based on the size of the PCR product and deciding that the theorem proving is true if a perfect double strand of DNA is formed.

6. The DNA computing method as defined in claim 4, wherein the size of the PCR product is measured with resort to polyacrylamide gel electrophoresis.

7. The DNA computing method as defined in claim, 1, wherein the DNA molecules corresponding to the clause are of branched structures and have single stranded ends representing literals.

8. The DNA conputing method as defined in claim 7, wherein each of the branch structures has as many arms as the literals contained in each clause and possesses a single-stranded base sequence end corresponding to a literal.

9. The DNA computing method as defined in claim 1, wherein the DNA molecules corresponding to the clauses are of hairpin structures ard have single stranded base sequence ends which corresponding to literals respectively.
